(19) United States
(12) Patent Application Publication
(10) Pub. No.: US 2010/0113620 A1
(43) Pub. Date: May 6, 2010

(54) ENTERIC PHARMACEUTICAL CAPSULES

(75) Inventors: Yvonne Perrie, Birmingham (GB); Alan Smith, Birmingham (GB)

Correspondence Address: MICHAEL BEST & FRIEDRICH LLP, 100 East Wisconsin Avenue, Suite 3300, Milwaukee, WI, 53202, US

(73) Assignee: ASTON UNIVERSITY, BIRMINGHAM (GB)

(21) Appl. No.: 12/593,440

(22) PCT Filed: Mar. 26, 2008
(86) PCT No.: PCT/GB08/01038

§ 371 Date: Sep. 28, 2009

(30) Foreign Application Priority Data
Mar. 29, 2007 (GB) 0706178.1

(51) Int. Cl. A61K 47/26; B29C 45/00

(52) U.S. Cl. 514/779; 264/328.1

(57) ABSTRACT

This invention pertains generally to the field of hard enteric capsules, and more particularly, to capsules consisting of (a) a film-forming, water-soluble polymer; (b) an acid-insoluble polymer; (c) a gelatinising agent; (d) an auxiliary for gelation; (e) at least one plasticiser; (f) minor ingredients, including, optionally, a colouring agent and a flavouring agent; and the balance of the composition being water. The present invention also pertains to the use of the capsule in the delivery of active pharmaceutical ingredients or dietary supplements and methods for producing hard enteric capsules.

The present invention relates to two-piece capsules for post-gastric delivery of pharmaceuticals following oral administration. The invention also provides a method for manufacturing such capsules. The invention further provides a polymer composition for pharmaceutical applications that can be manufactured without the incorporation of animal-based products, such as gelatin.

Encapsulation using a pre-moulded, two-piece hard capsule is just one of a multitude of drug delivery systems in practice and is extremely effective in protecting and delivering active pharmaceutical ingredients (APIs) or dietary supplements. Standard pharmaceutical hard capsules are generally manufactured from gelatin and are designed to dissolve in the stomach acid, releasing the drug, which is absorbed through the lining of the stomach. However, certain APIs are unsuitable for gastric release: certain drugs may irritate the gastric mucosa, be unstable or reactive at stomach acid pH, may interfere with gastric metabolism, or the drug target may be further along the GI tract.

If passage through the stomach for post-gastric delivery is required, there are two methods employed routinely:
1. Spray coat the loaded gelatin capsule with an acid-insoluble polymer, usually polymethacrylate-based EUDRAGIT™;
2. Spray coat the API with an acid-insoluble polymer prior to loading into a standard gelatin capsule.

However, there are two main problems associated with standard spray-coated gelatin capsules. Firstly, separate spray-coating procedures are costly and time-consuming, and problems such as uneven application, lack of adhesion, or cracking of the coating may arise, affecting the appearance and performance of the coating.

Secondly, there are a number of disadvantages associated with the use of gelatin in pharmaceutical hard capsules, arising from religious and ethical objections to the use of animal-based products in pharmaceuticals and possible reactions occurring between gelatin capsules and their contents (Digenis, at al., J. of Pharmaceutical Sciences, 83, 915-921 (1994)).

Capsules based on hydroxypropylmethyl cellulose (HPMC), rather than gelatin, are known in the art (see U.S. Pat. No. 6,517,865 and U.S. Pat. No. 5,431,917). However, none of these has bulk enteric properties and therefore these capsules need to be coated if they are to be used for post-gastric delivery. One-piece softgel capsules with bulk enteric properties are also known in the art (see WO 2004/030658). However, in many applications, hard two-piece capsules are preferable to one-piece softgel capsules since they allow the processes associated with shaping and filling the capsule to be simplified.

Currently, hard capsules are prepared industrially by dipping stainless steel mould pins into a solution of gelatin, and removing, inverting and drying them to form a film on the surface of the pin. The dried capsule films are then removed from the moulds, cut to the correct lengths, after which the caps and bodies are assembled, printed and packaged.

The aim of the present invention is to produce a hard two-piece capsules with bulk enteric properties.

It has been found that a polymer composition comprising a film-forming, water-soluble polymer (that need not be made from gelatin or other animal products), an acid-insoluble polymer, a gelatinising agent, an auxiliary for gelation, at least one plasticiser and, optionally, other minor ingredients such as colouring agents and flavouring agents, the balance of the composition being water, may be used to form hard capsules with bulk enteric properties that may be prepared according to methods well-known in the art. No sequestering agent is required in the composition.

Accordingly, a first aspect of the present invention provides a capsule consisting of a film-forming, water-soluble polymer, an acid-insoluble polymer, a gelatinising agent, an auxiliary for gelation, at least one plasticiser and minor ingredients, including, optionally, colouring agents and flavouring agents, the balance being water.

In a second aspect of the invention, capsules according to the first aspect may be manufactured by preparing a solution containing a film-forming, water-soluble polymer, an acid-insoluble polymer, a gelatinising agent, an auxiliary for gelation, at least one plasticiser, minor ingredients, including, optionally, colouring agents and flavouring agents, and no sequestering agent to form a gel mass, and producing capsules from the gel mass through a dip moulding process.

The capsules of the invention may be used to deliver active pharmaceutical ingredients or dietary supplements, and this is a further aspect of the invention, along with capsules containing such APIs or dietary supplements.

### BRIEF DESCRIPTION OF THE FIGURES

The Examples of the invention will be described with reference to the following figures.

FIGS. 1A to 1G shows the changes in G′ and G″ of capsule base material comparative formulations 1-3 (A-C) and formulations 1-4 (D-G) on cooling at 1° C./min (1 rad/s 0.5% strain).

### DETAILED DESCRIPTION OF THE INVENTION

The present invention relates to a capsule material based on a polymer composition containing a film-forming, water-soluble polymer, an acid-insoluble polymer, a gelatinising agent, an auxiliary for gelation, at least one plasticiser and, optionally, other minor ingredients such as colouring agents and flavouring agents, the balance of the composition being water. The polymer composition does not contain any sequestering agent.

Film-Forming Polymer

The film-forming polymer can be a cellulose derivative, such as hydroxypropylmethyl cellulose, hydroxyethyl cellulose, hydroxypropylcellulose, and hydroxyethylmethyl cellulose or another polysaccharide polymer, such as pullulan or starch.

Preferably, film-forming polymer is a cellulose derivative, and more preferably it is hydroxypropylmethyl cellulose. Preferably, the molecular weight of the hydroxypropylmethyl cellulose polymer is greater than 60 000, more preferably greater than 80 000. Preferably, the molecular weight of the hydroxypropylmethyl cellulose polymer is lower than 300 000. Preferably, the hydroxypropyl content of the hydroxypropylmethyl cellulose polymer lies between 4 and 20%, more preferably between 4 and 12%. Preferably, the methoxyl content of the polymer lies between 15 and 30%, more preferably between 20 and 30%.

The amount of film-forming polymer is preferably greater than 50 wt %, more preferably greater than 60 wt % of the capsule. The amount of film-forming polymer is preferably below 90 wt %, more preferably below 85 wt % of the capsule.

Acid-Insoluble Polymer

The acid-insoluble polymer can be selected from the group consisting of uronic acids, including mixtures thereof such as alginates; acrylic and methacrylic acid copolymers; cellulose acetate esters such as phthalate, butyrate, hydroxypropylmethyl cellulose phthalate; and salts thereof.

Preferably, the acid-insoluble polymer is alginate. The G:M ratio (guluronate to mannuronate ratio) of the alginate may be varied to affect the gelling properties of the alginate. Alginates with a G:M ratio of between 75:25 and 50:50 are particularly suitable for use in the present invention. A ratio of about 65:35 is preferred. The molecular weight of the alginate may vary from about 20,000 to about 150,000, and is preferably from about 30,000 to 50,000 and more preferably about 35,000.

The amount of acid-insoluble polymer is preferably greater than 1 wt %, more preferably greater than 2 wt % of the capsule. The amount of acid-insoluble polymer is preferably below 40 wt %, more preferably below 30 wt % of the capsule.

Gelatinising Agent

Suitable gelatinising agents include gellan gum, carrageenan, polysaccharide of tamarind seed, pectin, curdlan, gelatin, furcellaran, agar, agarose, agarose sulphate, alginates, chitosan, guar gum, locust bean gum, tara gum, gum Arabic, ghatti gum, Khaya grandifolia gum, tragacanth gum, karaya gum, pectin, Arabinan, xanthan, starch, Konjac mannan, galactomannan, funoran, acetan, welan, rhamsan, succinoglycan, scieroglycan, schizophyllan, pullulan, dextran and dextran sulphate. Preferably, the gelatinising agent is gellan gum, most preferably deacetylated gellan gum.

The amount of gelatinising agent is preferably greater than 0.1 wt %, more preferably greater than 0.2 wt % of the capsule. The amount of gelatinising agent is preferably below 1.5 wt %, more preferably below 2 wt % of the capsule.

Auxiliary for Gelation

The auxiliary for gelation is a compound containing monovalent or divalent cations such as K⁺, Na⁺, NH₄⁺, Ca²⁺, Mg²⁺, or Li⁺ and inert counterions, such as chloride. Preferably, the auxiliary for gelation contains sodium or potassium ions, most preferably sodium ions.

The amount of auxiliary for gelation is preferably greater than 0.1 wt %, more preferably greater than 0.2 wt % of the capsule. The amount of auxiliary for gelation is preferably below 1.5 wt %, more preferably below 2 wt % of the capsule.

Plasticisers

The polymer composition may include one or more suitable plasticisers, and in particular, those which are conventionally used in the pharmaceutical industry (see, for example, Pharmaceutical Capsules, ed. Podczeck, F. and Jones, B. E., Pharmaceutical Press, 2004, which is incorporated by reference). These plasticisers include polyethylene glycol, glycerol, sorbitol, sucrose, corn syrup, fructose, dioctyl-sodium sulfocuccinate, triethyl citrate, tributyl citrate, 1,2-propylenglycol, mono-, di-, or triacetates of glycerol, natural gums or the like as well as mixtures thereof.

Preferably, the plasticiser is polyethylene glycol, more preferably polyethylene glycol with a molecular weight between 150 and 250.

The amount of plasticiser is preferably greater than 5 wt %, more preferably greater than 10 wt % of the capsule. The amount of plasticiser is preferably below 25 wt %, preferably below 20 wt % of the capsule.

Colouring Agent

The polymer composition may also contain one or more colouring agents, and in particular, those which are conventionally used in the pharmaceutical industry (see, for example, Pharmaceutical Capsules, ed. Podczeck, F. and Jones, B. E.,

Pharmaceutical Press, 2004, which is incorporated by reference). These colouring agents include azo-, quinophthalone-, triphenylmethane-, xanthene-, or indigoid dyes, iron oxides or hydroxides, titanium dioxide or natural dyes or mixtures thereof.

The amount of colouring agent is preferably below 0.5 wt % of the capsule.

Flavouring Agents

The polymer composition may also contain one or more flavouring agents, and in particular those which are conventionally used in the pharmaceutical industry (see, for example, Pharmaceutical Capsules, ed. Podczeck, F. and Jones, B. E., Pharmaceutical Press, 2004, which is incorporated by reference). The flavouring substance may be any edible flavouring substance which is acceptable and approved for use with foods and/or pharmaceutical formulations. Very many such substances are known. The flavouring substance may for example be a natural or artificial flavouring, such as of a fruit, vegetable or confectionery taste. Examples of such flavourings are menthol, peppermint, and vanilla flavourings. If desired, more than one flavouring can be used in a single dosage means. Additionally or alternatively the flavouring substance may comprise a sweetener, such as sugar, sodium saccharin or aspartame.

The amount of flavouring agent is preferably below 0.5 wt % of the capsule.

Capsule Manufacture

The invention also relates to a process for manufacturing the hard capsules. This process includes preparing a solution containing a film-forming, water-soluble polymer, an acid-insoluble polymer, a gelatinising agent, and an auxiliary for gelation and mixing with appropriate plasticisers to form a gel mass. The colouring agents and flavouring agents (if present) may be added at the appropriate stage. No sequestering agent is required. Capsules may then be produced from the gel through a dip moulding or injection moulding process. Dip moulding involves dipping pins into the gel such that a film of gel forms on their surface. The pins are then inverted and the film is dried to form the capsule. The capsule films are then removed from the moulds and cut to the correct lengths. Injection moulding involves injection of the composition, which may be heated, into a mould, followed by allowing the composition to set, after which the capsule is removed from the mould.

Preferably, the solvent is water, most preferably de-ionised water. The solution is preferably formed at a temperature of 70° C. or more, more preferably 80° C. or more. The dip-moulding process is preferably carried out after cooling the solution to between 40 and 70° C., more preferably between 55 and 60° C. and the films are preferably allowed to dry on the pins for over 12 hours, more preferably over 24 hours.

After drying, the moisture content of the shell composition may lie below 20 wt %, more preferably below 10 wt %.

The invention also relates to a capsule composition having an acid-insoluble polymer to film-forming polymer ratio ranging from 1:18 to 1:2 by weight. Preferably this ratio is 1:3 or below.

### EXAMPLES

### Example 1

### Enteric Polymer Composition

Enteric polymer compositions were prepared from hydroxypropylmethylcellulose (HPMC) (Pharmacoat 606, Shin Etsu, Japan), alginate (Protonal LFR 5/60, FMC Biopolymer, Dramman, Norway—G:M ˜65:35; MW ˜35000), gellan gum (Gelrite, Kelco, Surrey, UK), an auxiliary for gelation (NaCl) (Sigma-Aldrich, Poole, UK). The gellan gum, alginate and NaCl were dissolved in deionised water at high temperature (>80° C.) before dispersing HPMC into the mixed polymer solution. The mixture was stirred for 2 hours until all material was fully hydrated and a homogenous dispersion was evident. This provided the base capsule material. Details of the formulations tested are given below:

| | HPMC | Gellan | Alginate | NaCl |
|---|---|---|---|---|
| Formulation | % w/w | % w/w | % w/w | % w/w |
| 1 | 18 | 0.2 | 1 | 0.2 |
| 2 | 17 | 0.2 | 2 | 0.2 |
| 3 | 15 | 0.2 | 5 | 0.2 |
| 4 | 15 | 0.2 | 7.5 | 0.2 |
| Comparative | 19 | 0 | 0 | 0 |
| formulation 1 | | | | |
| Comparative | 19 | 0.1 | 0 | 0.1 |
| formulation 2 | | | | |
| Comparative | 19 | 0.2 | 0 | 0.2 |
| formulation 3 | | | | |

### Example 2

### Rheological Analysis

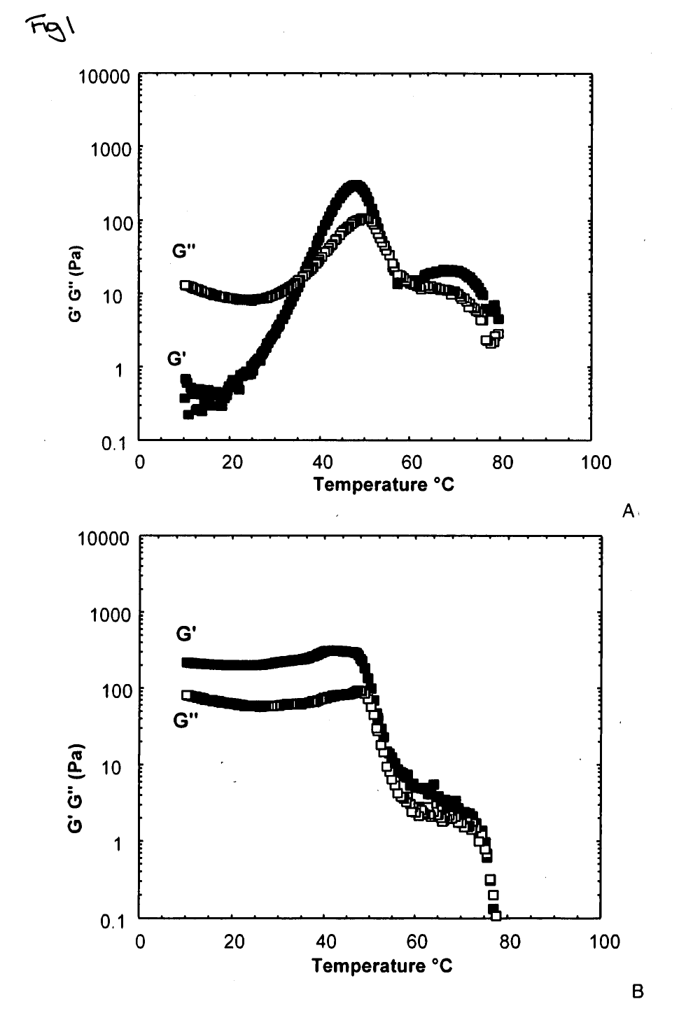

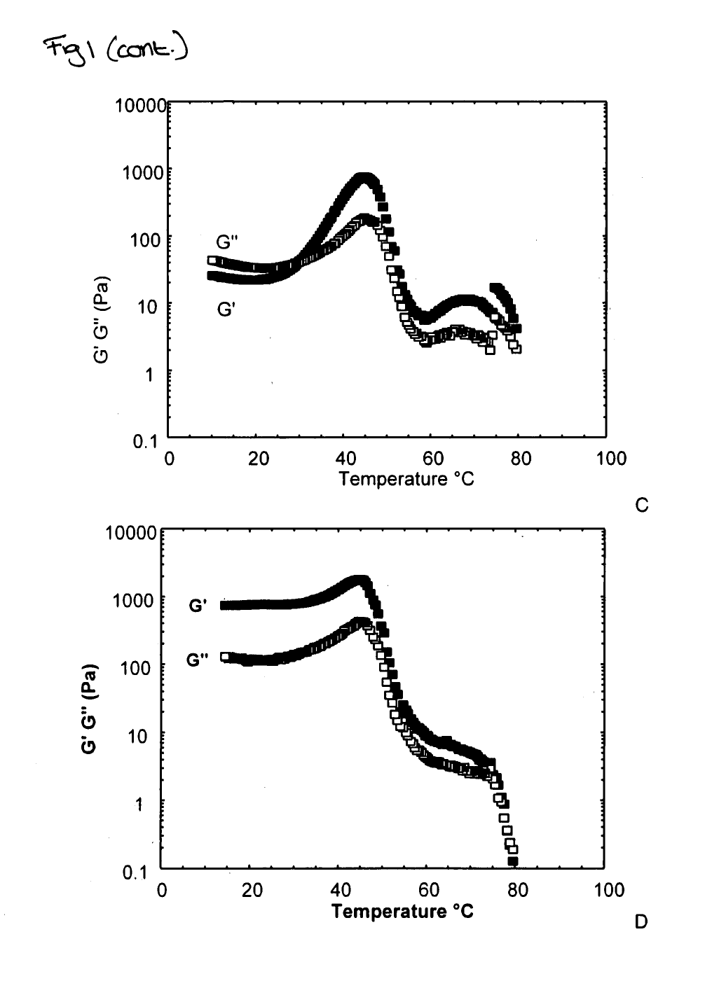

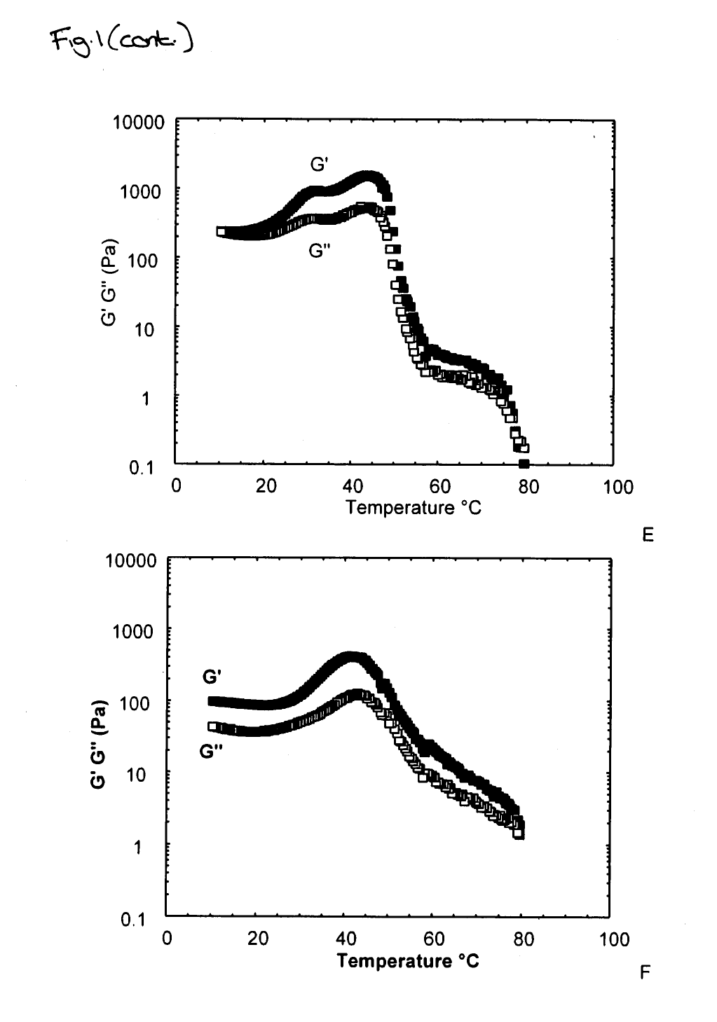

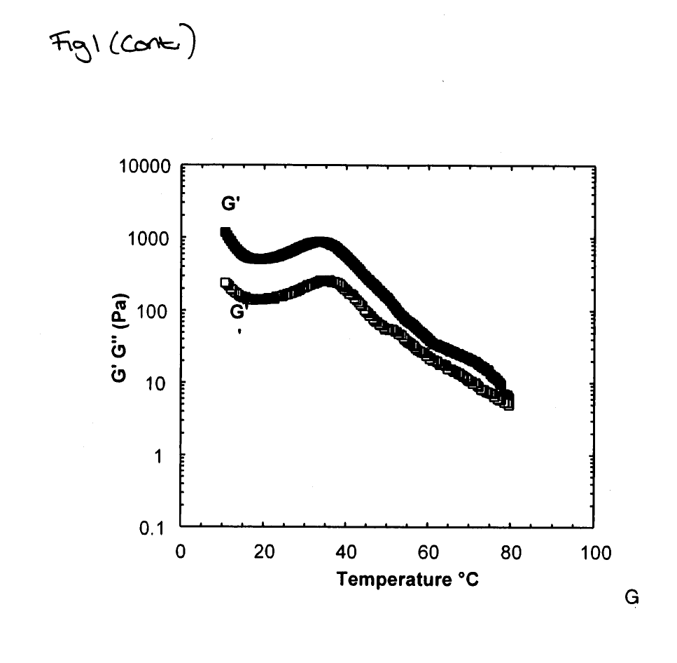

Rheological analysis of the capsule base formulations of Example 1 was carried out in the linear viscoelastic region using a 40 mm parallel plate geometry mounted on a Malvern Gemini Rheometer (Malvern Instruments, UK) fitted with peltier plate thermal control. Changes in G′ (elastic modulus) and G″ (viscous modulus) were measured during cooling at a rate of 1°/min performed at 0.5% strain over a temperature range 80-10° C., using a fixed oscillation frequency of 1 rad/s. The results are shown in FIG. 1. Formulations 1-4 and comparative formulation 2 exhibited G′>G″ at the end of the cooling process, indicating that a gel had been formed. Comparative formulations 1 and 3 exhibited G″>G′ at the end of the cooling process, indicating that the composition retained a strong liquid character.

### Example 3

### Dip Moulding

The polymer base mixtures of the formulations of Example 1 were cooled to 55-60° C. following preparation and a glass moulding pin was dipped into the mixture, removed and inverted to mimic the dip moulding process in industrial capsule production. Capsules were formed successfully for formulations 1-4 and comparative formulation 2.

It was not possible to form capsules from comparative formulations 1 and 3, as the liquid nature of these formulations caused the material to flow off the inverted moulding pin.

### Example 4

### Film Casting

The polymer base mixtures of the formulations of Example 1 were cooled to 55-60° C. following preparation, poured onto a Perspex plate and films were cast using a casting knife to produce films with a thickness in the range 0.12-0.15 mm. The films were allowed to dry for 24 hours.

### Example 5

### Puncture Tests

Puncture tests were carried out on 4 cm×4 cm film samples of Example 4 using texture profile analysis (TPA). A stainless steel probe was used to puncture at a rate of 10 mm/min. The maximum force achieved was recorded as the toughness parameter. The results of the tests are shown below.

| Formulation | Puncture Force (N) |
|---|---|
| 1 | 13.1 |
| 2 | 3.2 |
| 3 | 1.2 |
| 4 | N/A |
| Comparative formulation 1 | N/A |
| Comparative formulation 2 | 15.8 |
| Comparative formulation 3 | 19.2 |
| Comparative formulation 4 | 11.1 |
| (gelatin hard capsule) | |
| Comparative formulation 5 | 8.5 |
| (HPMC capsule) | |

The puncture force resistance of the formulations 2 and 3 is lower than that of capsules not containing alginate. The puncture resistance of formulation 4 was too low to be measured. However, replacing 5% of the water content of formulation with the plasticiser polyethylene glycol (PEG 200) results in an at least two-fold increase of the puncture resistance.

### Example 6

### Water Content Evaluation

Analysis of the water content of the polymer films of Example 4 was carried out by thermo-gravimetric analysis (TGA) (Perkin Elmer). A sample of film was heated at 10° C./min from 50° C. to 140° C. The water content was calculated from the reduction in mass on completion of the heating regime. The results of the tests are shown below.

| Formulation | Water content % weight |
|---|---|
| 1 | 9.8 |
| 2 | 8.7 |
| 3 | 7.0 |
| 4 | N/A |
| Comparative formulation 1 | N/A |
| Comparative formulation 2 | 6.5 |
| Comparative formulation 3 | 5.1 |
| Comparative formulation 4 | 10.5 |
| (gelatin hard capsule) | |
| Comparative formulation 5 | 4.5 |
| (HPMC capsule) | |

### Example 7

### Enteric Testing of Films

A circular piece of the polymer films of Example 4 (thickness: 250 μm; approximate diameter: 2 cm) was exposed to 0.1 M hydrochloric acid at 37° C. for 2 hours, followed by exposure to phosphate buffer at pH 6.8. The time taken for the buffer to pierce the film is given below.

| Formulation | Time taken for buffer to pierce film (min) |
|---|---|
| 1 | Film pierced in acid after one hour |
| 2 | 1 |
| 3 | 1 |
| 4 | N/A |
| Comparative Example 1 | N/A |
| Comparative Example 2 | Film pierced in acid after one hour |
| Comparative Example 3 | N/A |

1-9. (canceled)

10. A capsule consisting of
(a) a film-forming, water-soluble polymer
(b) an acid-insoluble polymer
(c) a gelatinising agent
(d) an auxiliary for gelation
(e) at least one plasticiser
(f) optionally, one or more colouring agents or flavouring agents,
the balance of the composition being water;
wherein the film-forming, water-soluble polymer is a cellulose derivative.

11. A capsule according to claim 10, wherein the cellulose derivative is hydroxypropylmethyl cellulose (HPMC).

12. A capsule according to claim 10, wherein the amount of film-forming polymer is greater than 50 wt % of the capsule.

13. A capsule according to claim 10, wherein the amount of film forming polymer is below 90 wt % of the capsule.

14. A capsule according to claim 10, wherein the amount of film-forming polymer is greater than 50 wt % and below 90wt % of the capsule.

15. A capsule according to claim 10, wherein the acid-insoluble polymer is alginate.

16. A capsule according to claim 10, wherein the gelatinising agent is gellan gum.

17. A capsule according to claim 10, wherein the auxiliary for gelation contains sodium or potassium ions.

18. A capsule according to claim 10, wherein the plasticiser is polyethylene glycol.

19. A capsule according to claim 10, wherein the gelatinising agent is gellan gum, and the auxiliary for gelation contains sodium or potassium ions.

20. A capsule according to claim 10, wherein the acid-insoluble polymer is alginate, the gelatinising agent is gellan gum, and the auxiliary for gelation contains sodium or potassium ions.

21. A capsule according to claim 10, wherein the acid-insoluble polymer is alginate, the gelatinising agent is gellan gum, the auxiliary for gelation contains sodium or potassium ions, and the plasticiser is polyethylene glycol.

22. A capsule according to claim 10, wherein the acid-insoluble polymer is alginate, the gelatinising agent is gellan gum, the auxiliary for gelation contains sodium or potassium ions, the plasticiser is polyethylene glycol, and the cellulose derivative is hydroxypropylmethyl cellulose (HPMC).

23. The use of a capsule according to claim 10, in the delivery of active pharmaceutical ingredients or dietary supplements.

24. A method for producing hard enteric capsules, comprising
(a) preparing a solution containing a film-forming, water-soluble polymer, an acid-insoluble polymer, a gelatinising agent, an auxiliary for gelation, at least one plasticiser and no sequestering agent to form a gel mass
(b) producing capsules through a dip moulding or injection moulding process; wherein the film-forming, water-soluble polymer in step (a) is a cellulose derivative.
